(19) United States
(12) Patent Application Publication
(10) Pub. No.: US 2025/0277229 A1
(43) Pub. Date: Sep. 4, 2025

(54) ABCG-TYPE SORGOLEONE TRANSPORTER FROM SORGHUM BICOLOR

(71) Applicants: The United States of America, as Represented by the Secretary of Agriculture, Washington, DC (US); DONALD DANFORTH PLANT SCIENCE CENTER, ST. LOUIS, MO (US); The Texas A&M University System, COLLEGE STATION, TX (US)

(72) Inventors: SCOTT R. BAERSON, OXFORD, MS (US); BAL K. MAHARJAN, COLLEGE STATION, TX (US); SAKIKO OKUMOTO, COLLEGE STATION, TX (US); VEENA VEENA, CHESTERFIELD, MO (US); JING XI, OXFORD, MS (US); CHARLES L. CANTRELL, Oxford, MS (US)

(21) Appl. No.: 18/591,213

(22) Filed: Feb. 29, 2024

(51) Int. Cl. C12N 15/82; A01N 31/16; A01P 13/00

(52) U.S. Cl. CPC C12N 15/8274; A01N 31/16; A01P 13/00; C12N 15/8227

(57) ABSTRACT

The present disclosure provides for the use of a sorgoleone transporter protein, originally from Sorghum bicolor, in transgenic organisms and cells, such as plants or plant cells. This transporter protein can be incorporated into any plant desired, either alone, or in combination with enzymes of the sorgoleone biosynthesis pathway. Thus, sorgoleone-producing plants and other cells can be constructed.

### SEQUENCE LISTING

The instant application contains a Sequence Listing XML required by 37 C.F.R. § 1.831(a) which has been submitted in XML file format via the USPTO patent electronic filing system and is hereby incorporated by reference in its entirety. The XML file was created on 02/28/2024, is named Sequence_Listing-006423.xml, and has 7.40 KB.

### BACKGROUND OF THE INVENTION

### Field of Invention

The present disclosure provides for the use of a sorgoleone transporter protein, originally from Sorghum bicolor, in transgenic organisms and cells, such as plants or plant cells. This transporter protein can be incorporated into any plant desired, either alone, or in combination with enzymes of the sorgoleone biosynthesis pathway. Thus, sorgoleone-producing plants, cells, and other organisms can be constructed.

### Background

Allelopathy, a form of chemical warfare between plants, can be defined as the production and release of chemical substances by one species that inhibit the growth of another species (Inderjit & Duke, Planta, (2003), 217:529-39; Weston & Duke, CRC Crit. Rev. Plant Sci., (2003), 22:367-89). Allelopathic interactions have been proposed to have profound effects on the evolution of plant communities through the loss of susceptible species via chemical interference, and by imposing selective pressure favoring individuals resistant to inhibition from a given allelochemical (Schulz & Wieland, Chemoecol., (1999), 9:133-41). Furthermore, allelopathic compounds released by grain crop species are thought to play a significant role in the utility of cover crops and intercropping systems where they act as weed suppressants. Allelopathic compounds have been characterized in a number of plants such as black walnut, wheat, rice, and sorghum (Bertin et al, Plant Soil, (2003), 256:67-83; Inderjit & Duke, supra; Duke S., Pest Manag. Sci., (2005), 61:211-18).

Despite the ecological and agronomic importance of allelochemicals, relatively few of the corresponding biosynthetic pathways have been characterized in detail at the molecular level. One notable exception is the identification and characterization of all the genes encoding the enzymes responsible for the biosynthesis of the benzoxazinoid 2,4-dihydroxy-7-methoxy-2H-1,4-benzoxazin-3(4H)-one in Zea mays (Frey et al, Science, (1997), 277:696-99). Benzoxazinoids are thought to act as allelopathic chemicals in the rhizosphere, in addition to being defense compounds against microbial pathogens and insect herbivores (Sicker et al, Int. Rev. Cytol., (2000), 198:319-46; Friebe, A., J. Crop Prod., (2001) 4:379-400).

The allelochemical sorgoleone represents an additional example where the biosynthetic pathway has been extensively characterized at both the biochemical and molecular level. Through the use of transcriptomics and biochemical evaluation of candidate sequences, all genes encoding enzymes required for the biosynthesis of sorgoleone, beginning with the ubiquitous precursor palmitoleoyl-CoA have now been identified (Pan et al, J. Biol. Chem., (2007); 282:4326-35; Baerson et al., J. Biol. Chem., (2008a) 283:3231-47; Pan et al, New Phytol., (2018), New Phytol., (2018) 218:616-29). The biosynthesis of sorgoleone occurs exclusively in root hairs, which appear as cytoplasmically dense cells in sorghum, containing large osmiophilic globules proposed to be associated with sorgoleone rhizosecretion (Czarnota et al, Weed Technol., (2001), 15:813-25; Czarnota et al, Int'l. J. Plant Sci., (2003), 164:861-66). The term sorgoleone is most frequently used to describe the compound corresponding to the predominant congener identified in sorghum root exudates (Netzly et al., Weed Sci., (1988), 36:441-6; Kagan et al., J. Agric. Food Chem., (2003), 51:7589-95), 2-hydroxy-5-methoxy-3-[(Z,Z)-8′,11′,14′-pentadecatriene]-p-benzoquinone, which has been estimated to account for between approximately 40-90% of the exudate material (w/w) in various accessions (e.g., Nimbal et al, J. Agric. Food Chem., (1996), 44:1343-47; Czarnota et al, (2001), supra; Baerson et al, (2008a), supra; Dayan et al., Plant Physiol., (2021), 187:1876-92). The remaining exudate consists primarily of 4,6-dimethoxy-2-[(Z,Z)-8′,11′,14′-pentadecatriene]resorcinol (methoxy-dihydrosorgoleone), and sorgoleone congeners differing in the length or degree of saturation of the aliphatic side chain, and in the substitution pattern of the quinone ring (Erickson et al, J. Agric. Food Chem., (2001), 49:5537-42; Kagan et al., supra; Rimando et al., J. Nat. Prod., (2003), 66:42-5; Dayan et al., supra). The fact that sorgoleone acts as a potent broad-spectrum inhibitor active against many agronomically important monocotyledonous and dicotyledonous weed species, and appears to affect multiple targets in vivo thus making it less susceptible to evolved weed resistance (Netzly & Butler, Crop Sci., (1986), 26:775-8; Einhellig & Souza, J. Chem. Ecol., (1992), 18:1-11; Nimbal et al, supra; Rimando et al, (1998) supra; Czarnota et al, (2001), supra; Bertin et al, (2003), supra; Duke, S., Trends Biotechnol., (2003), 21:192-5), make it promising for development as a natural product alternative to synthetic herbicides (Duke, S., (2003), supra). Sorgoleone has only been found to be produced by members of the genus Sorghum (Czarnota et al, J. Chem. Ecol., (2003b), 29:2073-83; Baerson et al, Plant Signal Behav., (2008b), 3:667-70).

One approach currently being pursued is the deployment of allelochemicals and other naturally occurring phytotoxins as plant-incorporated protectants (PIPs), defined as pesticides produced by plants via genetic modification. PIPs have been commercially released for both virus resistance and insect management (Mabubu et al, J. Entomol. Zool. Stud., (2016), 4:48-52; Niraula & Fodong, Plants, (2021), 10; 2339). By far the most commercially successful examples of plant-incorporated protectants are crops expressing Cry toxins derived from Bacillus thuringiensis (Bt). Beyond providing additional tools for insect management, Bt crops are environmentally beneficial, and benefit human health by significantly reducing the requirement for chemical insecticide treatments in crops. Furthermore, in 2009 global farm income was estimated to have increased by $2.9 billion following the wide-spread adoption of Bt crops, through a reduction in chemical insecticide expenditures as well as increased crop yields (Mabubu et al., supra). Despite the successful deployment of insect and virus-resistant crop varieties, currently no plant-incorporated protectant herbicides are available for weed management despite the obvious environmental and economic benefits such technologies could offer (Duke, S., Allelopathy J., (2010), 25:17-30).

Herein, we report the identification of an ATP Binding Cassette, subfamily G transporter from S. bicolor (genotype BTx623), designated SbABCG2, required for the rhizhosecretion of the phenolic lipid allelochemical sorgoleone. Also detailed herein are compositions and methods for constructing recombinant organisms and cells expressing this transporter, plants expressing this transporter, and plants expressing this transporter along with one or more enzymes of the sorgoleone biosynthesis pathway.

### SUMMARY OF THE INVENTION

The instant disclosure provides an expression vector containing, at a minimum, a promoter operatively linked to a heterologous polynucleotide that encodes a sorgoleone transporter at least 75%, 85%, or 95% identical to SEQ ID NO: 1. In some embodiments, the promoter is a constitutive promoter, a tissue-specific promoter (such as a root-hair-specific promoter), or an inducible promoter. In some embodiments, the heterologous polynucleotide has a sequence at least 75%, 85%, or 95% identical to SEQ ID NO: 2.

The instant disclosure further provides a cell transformed with the expression vector(s) described herein, where the cell is a plant cell, a fungal cell, a blue-green alga, and a bacterial cell. In some embodiments utilizing plant cells, the cell is not from Sorghum bicolor. Also disclosed are transgenic organisms comprising such transformed cells that produce a sorgoleone transporter with at least 75%, 85%, or 95% identity to SEQ ID NO: 1. In some embodiments, the transgenic organism is a plant. In many embodiments, the transgenic organism is not Sorghum bicolor.

The instant disclosure further provides a transgenic plant expressing a heterologous nucleic acid encoding a protein with at least 75%, 85%, or 95% identity to SEQ ID NO: 1. In many embodiments, such plants are not Sorghum bicolor.

The instant disclosure also provides a method of expressing a sorgoleone transporter in a plant or plant cell, by introducing a heterologous nucleic acid into a genome of the plant or plant cell, where the heterologous nucleic acid is operably linked to a constitutive, tissue-specific, or inducible promoter functional in the plant or plant cell, and the heterologous nucleic acid encodes a sorgoleone transporter at least 75%, 85%, or 95% identical to SEQ ID NO:1, and allowing the production of the sorgoleone transporter in the plant or plant cell. In some embodiments, the promoter is a tissue-specific promoter, such as a root-hair specific promoter. In most embodiments, the plant or plant cell is not Sorghum bicolor.

Further provided by the instant disclosure is a method of generating a sorgoleone-resistant plant, by introducing a heterologous nucleic acid into a genome of the plant or plant cell, where the heterologous nucleic acid is operably linked to a constitutive, tissue-specific, or inducible promoter functional in the plant or plant cell, and the heterologous nucleic acid encodes a sorgoleone transporter at least 75%, 85%, or 95% identical to SEQ ID NO:1, and allowing the production of the sorgoleone transporter in the plant or plant cell. In some embodiments, the promoter is a tissue-specific promoter, such as a root-hair specific promoter. In most embodiments, the plant or plant cell is not Sorghum bicolor.

Also provided by the instant disclosure is a method of controlling weeds in a field, comprising the steps of planting a transgenic plant expressing a heterologous sorgoleone transporter in the field, and applying sorgoleone to the field such that it contacts the weeds, thereby controlling sorgoleone-susceptible weeds.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1A and FIG. 1B depict analysis of S. bicolor genotype Tx430. FIG. 1A provides a comparison of sorgoleone contents in SX-17 (S. bicolor×S. sudanense hybrid) and S. bicolor genotype Tx430 seedlings. FIG. 1B Sorghum root hair cells isolated from seedlings grown in small-scale aeroponic containers, viewed under bright field microscopy at 100× magnification.

FIG. 2A, FIG. 2B and FIG. 2C depict chromosomal organization, tissue-specific expression, and transcriptional feedback repression of SbABCG2. FIG. 2A depicts “Sobic.003G215300”, the locus encoding ABCG2, is located on the reverse strand of chromosome 3 (S. bicolor v3.1.1 genome; phytozome-next.jgi.doe.gov) spanning nucleotides 54,994,919 to 55,002,839, containing 20 predicted exons. The arrows indicate the positions of the nucleotide transitions contained in two independent ems-generated mutant lines, each of which results in a stop codon. FIG. 2B depicts ABCG2 expression levels in root hair, root, immature leaf (I-leaf), mature leaf (M-leaf), stem, apex and panicle as determined by RNA-seq analysis. FIG. 2C depicts ABCG2 expression levels in sorgoleone-deficient transgenic seedlings (278-RNAi) versus null segregant seedlings derived from the same transformation event (278-null), as determined by RNA-seq analysis.

FIG. 3A and FIG. 3B depict data showing SbABCG2 is required for sorgoleone rhizosecretion. FIG. 3A provides micrographs showing abcg2-1 and abcg2-2 EMS mutants lack conspicuous root hair secretion visible in WT BTx623 seedlings. FIG. 3B provides representation of data from HPLC quantification of sorgoleone content in WT and abcg2-1 plants.

FIG. 4 provides pictorial representation of an exemplary vector for creation of a plant expressing heterologous SbABCG2.

FIG. 5 provides pictorial representation of the sorgoleone biosynthesis pathway, including the enzymes and transport protein (SbABCG2).

### DETAILED DESCRIPTION OF THE INVENTION

Disclosed herein is the use of isolated nucleic acids encoding a sorgoleone transporter (SbABCG2) derived from S. bicolor to create transgenic organisms, particularly plants or plant cells, expressing this transporter. In most embodiments, the transgenic organism is not Sorghum bicolor. In some embodiments, transgenic plants expressing the SbABCG2 protein gain resistance to the herbicidal activity of sorgoleone. In some embodiments, expression of SbABCG2 in plant cells can be used as a selectable marker by conferring resistance to sorgoleone. This disclosure also provides methods of making plants capable of secreting sorgoleone by transforming the plant with one or more enzymes of the sorgoleone biosynthesis pathway in addition to the transporter.

The in planta production of an effective PIP herbicidal compound requires both the biosynthesis and correct delivery of the compound to its final site of action. Allelochemicals are typically exuded from plant root systems where they can effectively inhibit the growth and development of neighboring plants. Currently a paucity of information exists concerning the transport mechanisms facilitating the secretion of plant allelochemicals into the rhizosphere, however in the case of sorgoleone its chemical structure may provide some clues. Sorgoleone, an alkyresorcinol derivative belonging to the phenolic lipid family of compounds, is produced through the action of unusual type III polyketide synthases utilizing hexadecatrienoyl-CoA (C16:3Δ^{9,12,15}) starter units (SbARS1,2; Cook et al 2010). Sorgoleone represents one of the more extensively-studied phenolic lipids identified in plants; other important examples include urushiol, an allergen from poison ivy (Toxicodendron radicans), anacardic acid, an anti-feedant found in several dicotyledonous species such as cashew (Anacardium occidentale), as well as the alkylresorcinol phytoanticipins found throughout the Poaceae (grass) family (Kozubek & Tyman, Chem. Rev., (1999), 99:1-26; Kozubek et al., Cell. Mol. Biol., (2001), 6:351-5). Plant-derived phenolic lipids have also been used by industry, for example in the manufacturing of formaldehyde-based polymers and in lacquering processes (Kozubek & Tyman, supra).

ATP Binding Cassette transporters represent a superfamily of integral membrane transporters found in both eukaryotic and prokaryotic organisms, which use energy derived from ATP hydrolysis to drive the translocation of diverse substrates across cellular membranes (Do et al, Plant Physiol., (2021), 187:1876-92). ABC proteins are divided into 8 subfamilies (A, B, C, D, E, F, G and I), and in Arabidopsis thaliana the ABCG subfamily contains the most members (Gräfe & Schmitt, J. Exp. Bot., (2021), 72:92-106). Most ABC proteins contain 2 variable transmembrane domains and 2 highly conserved nucleotide binding domains which constitute functional transporters, however half-size ABC proteins possessing a single nucleotide binding domain and transmembrane domain also occur and assemble as hetero- or homodimers for their functionality (Li et al., Trends Plant Sci., (2016), 21:145-58). A signatory structural feature of G subfamily proteins is the positioning of a conserved nucleotide binding domain near the N-terminus. In animals and prokaryotes only half-size ABCG proteins have been identified to date, hence full-size ABCGs (also referred to as pleiotropic drug resistance or PDR proteins) are likely restricted to plants, fungi, slime molds, oomycetes, and brown algae (Grafe & Schmitt, supra).

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This invention teaches methods and describes tools for the construction of vectors, including expression vectors, encoding a sorgoleone transporter and the construction of recombinant organisms expressing a sorgoleone transporter, such as one having high similarity to SEQ ID NO:1.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

The term “a nucleic acid consisting essentially of”, and grammatical variations thereof, means nucleic acids that differ from a reference nucleic acid sequence by 20 or fewer nucleic acid residues and also perform the function of the reference nucleic acid sequence. Such variants include sequences which are shorter or longer than the reference nucleic acid sequence, have different residues at particular positions, or a combination thereof.

“Control” or “controlling” as used herein refers to means for preventing growth in a treated area (e.g., of a weed in a field), reducing the population of already affected areas, or elimination of a population(s) whose “control” is desired. Indeed, “controlling” as used herein refers to any indicia of success in prevention, elimination, reduction, repulsion, or amelioration of a weed population or weed problem.

The term “SbABCG2” refers to the protein defined herein as SEQ ID NO:1 and encoded by the DNA of SEQ ID NO:2 (or any version of SEQ ID NO:2 with base substitutions that result in a protein with a sequence identical to SEQ ID NO:1).

A “genetically altered” organism is any organism with any changes to its genetic material (as compared to an unmodified organism), whether in the nucleus or cytoplasm (mitochondria and plastids). As such, a genetically altered organism can be a recombinant or transformed organism. Also, an organism that has been bred to incorporate a mutation into its genetic material is a genetically altered organism.

A “heterologous” protein or polypeptide refers to a protein or polypeptide derived from a different source or species than the cell or organism comprising or expressing the protein. Similarly, a heterologous nucleic acid is one not naturally part of the genome, or other genetic components associated with the organism (e.g., mitochondrial genome, plasmids, etc.).

A first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. Multiple nucleic sequences can be operably linked together, for example a promoter can be operably linked to a coding sequence which is operably linked to a terminator sequence.

The term “plant” includes whole plants, plant organs, and progeny of same. Plant organs comprise, e.g., shoot vegetative organs/structures leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like).

The techniques and approaches described herein can be applied to transform a wide variety of plants, including decorative or recreational plans or crops, but are particularly useful for treating commercial and ornamental crops. Other suitable hosts include blue-green algae and other photosynthetic organisms, even if these organisms are not considered plants. The class of plants that can be used to practice the instant disclosure is generally as broad as the class of higher and lower plants amenable to transformation techniques, including angiosperms (monocotyledonous and dicotyledonous plants), C3 plants, C4 plants, gymnosperms, ferns, and multicellular algae, Plants utilizable in practicing the disclosure herein can have a variety of ploidy levels, including, but not limited to, aneuploid, polyploid, diploid, haploid and hemizygous.

As used herein, the term “promoter” refers to a polynucleotide that in its native state is located upstream or 5′ to a translational start codon of an open reading frame (or protein-coding region) and that is involved in recognition and binding of RNA polymerase and other proteins (trans-acting transcription factors) to initiate transcription. The term can include promoters produced through the manipulation of known promoters to produce artificial, chimeric, or hybrid promoters. Such promoters can also combine cis-elements from one or more promoters, for example, by adding a heterologous regulatory element to an active promoter with its own partial or complete regulatory elements. The term “cis-element” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact with different affinities with more than one cis-element. Promoters include constitutive promoters, activatable promoters, tissue-specific promoters (such as root-hair-specific promoters), plant-specific promoters, and others that the skilled artisan would recognize as useful in practicing the instant disclosure.

The term “recombinant” when used with reference, e.g., to a cell, or nucleic acid, protein, or vector, indicates that the cell, organism, nucleic acid, protein or vector, has been modified by the introduction of a heterologous nucleic acid or protein or the alteration of a native nucleic acid or protein, or that the cell is derived from a cell so modified. Thus, for example, recombinant cells may express genes/polynucleotides that are not found within the native (non-recombinant or wild-type) form of the cell or express native genes in an otherwise abnormal amount—over-expressed, under-expressed or not expressed at all—compared to the non-recombinant or wild-type cell or organism. In particular, one can alter the genomic DNA of a wild-type plant by molecular biology techniques that are well-known to one of ordinary skill in the art to generate a recombinant plant.

The phrase “high percent identical” or “high percent identity”, and grammatical variations thereof in the context of two polynucleotides or polypeptides, refers to two or more sequences or sub-sequences that have at least about 70%, identity, at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% nucleotide or amino acid identity, when compared and aligned for maximum correspondence, as measured using a sequence comparison algorithm or by visual inspection.

The terms “transgenic”, “transformed”, “transformation”, and “transfection” are similar in meaning to “recombinant”. “Transformation”, “transgenic”, and “transfection” refer to the transfer of a polynucleotide into a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur the organism or cell may need to be kept under certain conditions. Genetically altered organisms or cells containing the recombinant polynucleotide can be referred to as “transgenic” or “transformed” organisms or cells or simply as “transformants”, as well as recombinant organisms or cells.

A “vector” is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An “expression vector” is a vector that contains the necessary regulatory sequences to allow transcription and translation of inserted gene or genes.

### Sorgoleone Biosynthesis Pathway

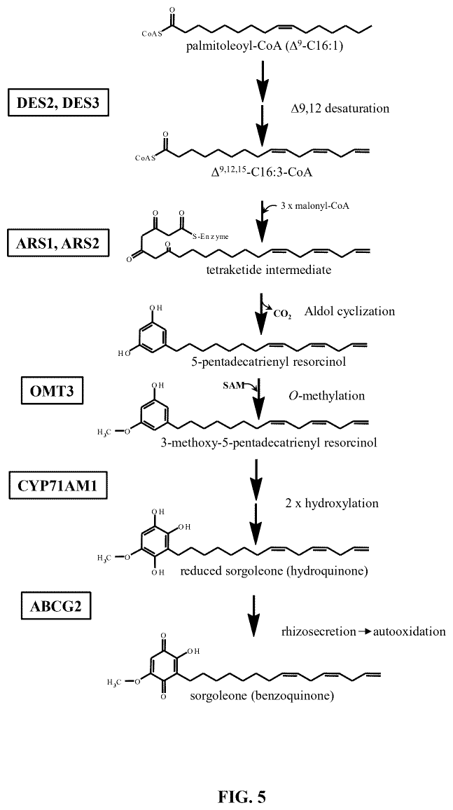

Molecular and biochemical investigations have shed significant light on the genes and corresponding enzymes associated with sorgoleone biosynthesis (Weston et al, J. Chem. Ecol., (2013), 39:142-53; Pan et al, (2018), supra; FIG. 5). Sorgoleone belongs to a family of compounds referred to as phenolic lipids, which have been identified in numerous plant, fungal, and bacterial taxa, but relatively few animal species. Among the major classes of phenolic lipids, which include alkylphenols, alkylresorcinols, anacardic acids, and alkylcatechols, the alkylresorcinols are by far the most prevalent in nature (Kozubek & Tyman, “Bioactive phenolic lipids”. In: Rahman A, ed., Studies in Natural Products Chemistry. Amsterdam: Elsevier B V; 2005. pp. 119-190.). The synthesis of sorgoleone and other phenolic lipids had long been assumed to occur via the action of specialized type III polyketide synthase (PKS) enzymes utilizing atypical fatty acyl-CoA starter units. In vivo labeling studies performed by Fate and Lynn (J. Am. Chem. Soc., (1996), 118:11369-76) provided the first evidence that this was likely the case for sorgoleone biosynthesis, although definitive proof for this concept was finally obtained following the isolation of type III PKSs from S. bicolor (designated ARS1 and ARS2; alkylresorcinol synthase) possessing alkylresorcinol-forming activity (Weston et al, (2013), supra). Additionally, gene knockdown experiments using RNA interference targeting ARS1 and ARS2 in transgenic sorghum plants resulted in multiple independent transformation events exhibiting dramatically reduced or undetectable levels of sorgoleone, thus providing unambiguous proof for the involvement of ARS1 and ARS2 in sorgoleone biosynthesis.

As mentioned, sorgoleone biosynthesis appears to occur exclusively or primarily in sorghum root hair cells, and further support for this notion comes from the observation that the 5-n-pentadecatrienyl resorcinol biosynthetic intermediate as well as the A9,12,15-C16:3 fatty acid used to generate the starter acyl-CoA used for its production accumulate only within this cell type (Weston et al., (2013), supra). Furthermore, the proposed sequence of tailoring reactions, where O-methylation precedes the addition of two hydroxyl groups within the ring moiety is supported by the identification of 3-methoxy-5-n-pentadecatrienyl resorcinol within S. bicolor root tissues.

One of the most highly expressed sequences identified in S. bicolor genotype BTx623 root hair cells (representing approximately 0.46% of all ESTs in this cell type) was also identified as a root hair-specific fatty acid desaturase-like sequence, and subsequent recombinant enzyme studies in S. cerevisiae revealed that this desaturase (designated DES3) could catalyze the formation of Δ^{9,12,15}-C16:3 fatty acid from hexadecadienoic acid (16:2 Δ^{9,12}). In the S. cerevisiae expression system employed, co-expression of DES3 with a second fatty acid desaturase enzyme identified within the EST data set (designated DES2) was required to generate hexadecadienoic acid from the endogenous palmitoleic acid pool (16:1 Δ⁹), thus providing the substrate used by recombinant DES3 for generating the Δ^{9,12,15}-C16:3 fatty acid. In contrast to other plant fatty acid desaturases reported to date, the double bond introduced by DES3 occurs between carbons 15 and 16 of the Δ^{9,12}-C16:2 precursor, resulting in the formation of an unusual terminal double bond in the resulting fatty acid (Weston et al, (2013), supra).

The 5-pentadecatrienyl resorcinol sorgoleone biosynthetic intermediate produced via alkylresorcinol synthase action in planta is likely next methylated by a root hair-specific O-methyltransferase, also identified within the BTx623 root hair data set (designated OMT3; (Weston et al, (2013), supra)). When tested in vitro by enzymatic assays, recombinant OMT3 was found to exhibit a marked preference for alkylresorcinolic substrates among a panel of diverse phenolic substrates analyzed, strongly suggesting involvement in the sorgoleone biosynthetic pathway. The final enzyme-mediated reactions involve the di-hydroxylation of 5-pentadecatrienyl resorcinol-3-methyl ether at the two ortho positions (C4 and C6) relative to the aliphatic side chain, resulting in the conversion of the intermediate into dihydrosorgoleone (a hydroquinone: 5-methoxy-3-((8Z,11Z)-pentadeca-8,11,14-trien-1-yl)benzene-1,2,4-triol), the direct precursor to sorgoleone (Pan et al, (2018), supra). The chemically unstable hydroquinone oxidizes upon rhizosecretion to the bioactive benzoquinone sorgoleone, which can persist in soil for extended periods (Weston et al, (2013), supra). A root hair-specific bifunctional cytochrome P450 monooxygenase was recently identified which can convert 5-pentadecatrienyl resorcinol-3-methyl ether to dihydrosorgoleone when heterologously expressed in S. cerevisiae, performing hydroxylation at both the 4′ and 6′ positions within the aromatic moiety. RNAi-mediated repression of the corresponding sequence in S. bicolor transformants resulted in decreased sorgoleone contents in multiple independent events, thus demonstrating a role for this P450 enzyme in sorgoleone biosynthesis (Pan et al., 2018). The enzyme is positioned within a subfamily of the plant-specific CYP71 clan and has been designated CYP71AM1. Importantly, the S. bicolor ABCG-type transporter described herein could not only be utilized in combination with the full complement of sorgoleone pathway enzymes (described above) in transgenic plants, but could also be potentially used for the cellular export of biologically active alkylresorcinols such as the direct enzymatic products of alkylresorcinol synthases ARS1 and ARS2.

### Molecular Biological Methods

An isolated nucleic acid is a nucleic acid the structure of which is not identical to that of any naturally occurring nucleic acid. The term therefore covers, for example, (a) a DNA which has the sequence of part of a naturally occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transformed or transfected cells, and (iii) cell clones, e.g., as these occur in a DNA library such as a cDNA or genomic DNA library.

Recombinant nucleic acids include polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

In practicing some embodiments of the instant disclosure, it can be useful to modify the genomic DNA, chloroplast DNA or mitochondrial DNA of a recombinant strain of a host cell to produce SbABCG2 and/or proteins of the sorgoleone biosynthesis pathway to introduce genetic elements allowing for the expression of introduced genes (e.g., promoters and other regulatory elements). In some embodiments, such a host cell is a plant cell.

Where a recombinant nucleic acid is intended for expression, cloning, or replication of a particular sequence, DNA constructs prepared for introduction into a host cell will typically comprise a replication system (i.e., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., chloroplast localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Screening and molecular analysis of modified organisms (e.g., transgenic plants, transgenic plant cells, or recombinant bacteria) of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al, (1989) (supra) or Ausubel et al, (1995) Current Protocols in Molecular Biology, John Wiley & Sons, NY, N.Y., for further guidance on hybridization conditions.

Nucleic acids and proteins of the present disclosure can also encompass homologues of the specifically disclosed sequences. Homology (e.g., sequence identity) can be 50%-100%. In some instances, such homology is greater than 70%, greater than 75%, greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin & Altschul, Proc. Natl. Acad. Sci., U.S.A., (1990), 87:2264-2268, modified as in Karlin & Altschul, Proc. Natl. Acad. Sci., U.S.A., (1993), 90:5873-5877. Such an algorithm is incorporated into the NBLAST and XBLAST programs of Altschul et al, J. Mol. Biol., (1990), 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al., Nucl. Acids Res., (1997), 25:3389-3402. When utilizing BLAST and Gapped BLAST programs, the default parameters of the respective programs (NBLAST and XBLAST) are used. See www.ncbi.nih.gov.

Recombinant host cells (such as transgenic plant cells or recombinant microbial cells), in the present context, are those which have been genetically modified to contain an isolated nucleic molecule, and/or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

### Transgenic Plants and Plant Cells

One embodiment of the present disclosure provides a plant or plant cell comprising a heterologous gene encoding SbABCG2. Such a gene typically would encode a protein identical to SEQ ID NO:1, such as by the sequence of SEQ ID NO:2. In some embodiments, variants of these sequences can be utilized, as long as the protein produced by the transgenic host retains the ability to transport sorgoleone. Such variants of SEQ ID NO:1 or SEQ ID NO:2 can be 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to those reference sequences. Any heterologous genes of the instant disclosure can be introduced into the host cell operably linked with regulatory sequences (e.g., promoter) or can be introduced into a host cell genome so as to create an operable linkage between a native promoter and the heterologous gene(s).

In specific embodiments, this disclosure provides for transgenic plant cells and transgenic plants expressing a heterologous sorgoleone transporter that's at least 90-100% identical to the sorgoleone transporter (SbABCG2) having SEQ NO:1. In some embodiments, the plant is not a member of the species Sorghum bicolor. In some embodiments, such transgenic plants can also comprise one or more sorghum biosynthesis enzymes.

Transformation and generation of genetically altered monocotyledonous and dicotyledonous plant cells is well known in the art (see, e.g., Weising, et al., Ann. Rev. Genet., (1988), 22:421-477; see, also, U.S. Pat. No. 5,679,558). The choice of method varies with the type of plant to be transformed, the particular application, and/or the desired result. The appropriate transformation technique is readily chosen by the skilled practitioner.

Any methodology known in the art to delete, insert or otherwise modify cellular DNA (e.g., genomic DNA and organelle DNA) can be used in practicing the inventions disclosed herein. For example, a disarmed Ti-plasmid, containing a genetic construct for deletion or insertion of a target gene, in Agrobacterium tumefaciens can be used to transform a plant cell, and thereafter, a transformed plant can be regenerated from the transformed plant cell using procedures described in the art, (see, e.g., EP 0116718, and EP 0270822). Ti-plasmid vectors each contain the gene between the border sequences, or at least located to the left of the right border sequence, of the T-DNA of the Ti-plasmid. Of course, other types of vectors can be used to transform the plant cell, using procedures such as symbiont technology (e.g., WO 21/055656), direct gene transfer (e.g., EP 0233247), pollen mediated transformation (e.g., U.S. Pat. No. 4,684,611), plant RNA virus-mediated transformation (e.g., U.S. Pat. No. 4,407,956), liposome-mediated transformation (e.g., U.S. Pat. No. 4,536,475), and other methods such as the methods for transforming certain lines of corn (e.g., U.S. Pat. No. 6,140,553) and rice (Shimamoto et al., Nature, (1989) 338, 274-276; Datta et al., Bio/Technology, (1990) 8, 736-740) and for transforming monocots generally (WO 92/09696). Genome editing technologies, such as CRISPR/Cas have been used extensively in a wide variety of plant species (see, e.g., Impens et al, Front. Genome Edit., (2022), 4:825042).

Transgenic plants of the present disclosure can be used in a conventional plant breeding scheme to produce more transgenic plants with the same characteristics, or to introduce the genetic alteration(s) in other varieties of the same or related plant species. Seeds, which are obtained from the transformed plants, can contain the genetic alteration(s) as a stable insert in chromosomal or organelle DNA. Plants comprising the genetic alteration(s) in accordance with the disclosure include plants comprising, or derived from, root stocks of plants comprising the genetic alteration(s) of the disclosure, e.g., fruit trees or ornamental plants. Hence, any non-transgenic grafted plant parts inserted on a transformed plant or plant part are included in the disclosure.

Introduced genetic elements which result in the expression of an introduced gene will typically utilize a plant-expressible promoter. A ‘plant-expressible promoter’ as used herein refers to a promoter that ensures expression of the genetic alteration(s) of the disclosure in a plant cell. Non-limiting examples of promoters directing constitutive expression in plants are known in the art and include: Sorghum-derived promoters (e.g., U.S. Pat. Nos. 10,000,762 and 10,815,491), the strong constitutive 35S promoters of the cauliflower mosaic virus (CaMV), e.g., of isolates CM 1841 (Gardner et al., Nucleic Acids Res, (1981) 9, 2871-2887), CabbB-S (Franck et al., Cell (1980) 21, 285-294) and CabbB-JI (Hull and Howell, Virology, (1987) 86, 482-493); promoters from the ubiquitin family (e.g., the maize ubiquitin promoter of Christensen et al., Plant Mol Biol, (1992), 18:675-689), the gos2 promoter (de Pater et al., Plant J., (1992), 2:834-844), the emu promoter (Last et al., Theor. Appl. Genet., (1990), 81:581-588), actin promoters such as the promoter described by An et al. (Plant J., (1996), 10:107), the rice actin promoter described by Zhang et al. (Plant Cell, (1991), 3:1155-1165); promoters of the Cassava vein mosaic virus (e.g., WO 97/48819), the pPLEX series of promoters from Subterranean Clover Stunt Virus (e.g., WO 96/06932), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the F and 2′ genes, respectively, of the T-DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in root hairs (see, e.g., U.S. Pat. Nos. 10,000,762 and 10,815,491). Other examples of tissue-specific promoters are known in the art, including but not limited to, green tissues (e.g., the promoter of PEP carboxylase; Pathirana et al., Plant J., (1997), 12:293-304) and wound-inducible promoters (e.g., Zhang et al., Plant Physiol, (1996), 112:1111-1117).

In some embodiments, genetic elements can be used to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene (e.g., SbABCG2) of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al, J. Mol. Appl. Gen., (1982), 1:561-573), the octopine synthase gene (Gielen et al, EMBO J., (1984), 3:835-845), the SCSV or the Malic enzyme terminators (Schunmann et al., Plant Funct. Biol., (2003), 30:453-460), and the T-DNA gene 7 (Velten & Schell, Nucl. Acids Res., (1985),13:6981-6998), which act as 3′-untranslated DNA sequences in transformed plant cells.

The embodiments illustratively disclosed herein suitably may be practiced in the absence of any element (e.g., method (or process) steps or composition components) which is not specifically disclosed herein. Thus, the specification includes disclosure by silence. Written support for a negative limitation may also be found through the absence of the excluded element in the specification, known as disclosure by silence.

### Application of Sorgoleone to Control Non-Resistant Plants

The sorgoleone-transporting transgenic plants of the present invention gain resistance to sorgoleone, thus allowing for the use of sorgoleone as a broad-spectrum herbicide to control undesired plant species (e.g., weeds) in a target area. In some embodiments, such transgenic plants also contain the enzymes of the sorgoleone biosynthesis pathway, thus produce sorgoleone and excrete it into the environment.

In additional embodiments, sorgoleone can be applied to fields, weeds, soil, and any other desired target using any delivery methodology known to those of skill in the art. For example, sorgoleone-containing compositions can be applied to a desired locale via methods including sprays, granules, flood/furrow methods, sprinklers, fumigation, root soaking and drip irrigation. Where the compositions are sprayed onto a desired locale, the compositions can be delivered as a liquid suspension, emulsion, microemulsion or powder. In other embodiments, granules or microcapsules can be used to deliver the compositions of the invention.

Sorgoleone compositions can be applied to plants and/or crop fields by any convenient method, for example, by using a fixed application system such as a center pivot irrigation system. Preferably, application to fields of plants and/or crops is made by air spraying, i.e., from an airplane or helicopter, or by land spraying. For example, land spraying may be carried out by using a high flotation applicator equipped with a boom, by a back-pack sprayer or by nurse trucks or tanks. One of skill in the art will recognize that these application methodologies are provided by way of example and that any applicable methods known in the art or developed in the future can be utilized.

Having generally described this invention, the same will be better understood by reference to certain specific examples, which are included herein to further illustrate the invention and are not intended to limit the scope of the invention as defined by the claims.

### EXAMPLES

### Example 1

### Identification of a Full-Length ABC Subfamily G Protein Member Required for Sorgoleone Rhizosecretion

### Materials and Methods

### Plant Materials and Growth Conditions

Seeds of Sorghum bicolor genotype BTx623 were purchased from Crosbyton Seed Co. (Crosbyton, TX, USA). Root tissues used for sorgoleone content determinations, and root hair preparations and whole root systems used for tissue-specific RNA-seq experiments were obtained from 5- or 8-day-old dark-grown seedlings grown under soil-free conditions using a capillary mat system devised by Czarnota and co-workers (Czarnota et al., (2003b), supra). Immature leaves and shoot apices were isolated from seedlings maintained in a growth chamber at 28° C. for 8 days in standard (approximately 20×40 cm) nursery flats using Premier Pro Mix PGX potting media (Hummert International, Earth City, MO) under a combination of cool-white fluorescent and incandescent lighting at an intensity of approximately 400 μmol m² s⁻¹ and a 16-h photoperiod. Developing panicles, mature leaves, and stem tissues used for tissue-specific RNA-seq studies were isolated from 10-week-old greenhouse-grown plants. At the time of harvest, panicles were partially exerted from flag leaf sheaths, just prior to anthesis. Root tissues for root hair preparations used in sorgoleone-deficient vs. null and wild-type RNA-seq experiments were obtained from 7-day-old dark-grown seedlings grown in Phytatra III trays (Millipore Sigma, St. Louis, CO, USA). Briefly, seeds were surface sterilized with bleach solution (2.5% NaOCl, 0.02% Triton X-100) for 30 min and rinsed six times with distilled water prior to planting. Three pieces of pre-cut Whatman #1 filter paper soaked with sterile 0.25X MS media (PhytoTech Labs, Lenexa, KS, USA) were placed beneath one piece of pre-cut Weed-X landscape fabric (Hummert International, Earth City, MO, USA) in the bottom of a Phytatray. Twenty seeds were placed on the surface of the Weed-X in each tray. A sterile, perforated stainless-steel sheet (McNichols Co. Atlanta, GA, USA) was placed on top of the seeds. Trays were maintained placed in a Percival growth chamber (29° C., dark-grown) for 8 days, and fresh 0.25X MS solution was added as needed. All harvested plant materials were directly flash-frozen in liquid nitrogen and stored at −80° C. prior to use.

### Root Exudate Collection

Root exudates were collected by first immersing roots in dichloromethane (DCM) acidified with 0.25% glacial acetic acid for ˜10 min. The DCM crude extract was then decanted through a porcelain Buchner funnel lined with Whatman #42 filter paper to remove root debris. The filtrated extracts were concentrated using a rotary evaporator (Buchi, New Castle, DE, USA) with water bath temperature set at 40° C. The dry extracts appeared as yellow residues and were transferred to a pre-weighed glass vial and dried under nitrogen. The dry weights of root exudates were obtained by weighing the vial containing the dry extract and subtracting the weight of the empty vial. Dried extracts were stored at −20° C. prior to HPLC quantification of sorgoleone.

### HPLC Determination of Sorgoleone Content

Sorgoleone HPLC quantitative analysis was performed using an Agilent 1260 series system (Agilent 1200 series consisting of a vacuum degasser, quaternary pump, ALS autosampler, and a diode array detector) and an Agilent Eclipse XDB-C18, 4.6 mm×150 mm, 5 μm column. Injection volume for all samples and for the sorgoleone standard was 10 μL. The analytical method was isocratic (28% acetonitrile: 72% deionized water with 0.1% TFA) for 20 min. Analytes were detected at 220 nm. Sorgoleone standard used for quantitative analysis was isolated and purified previously by our group and identified by comparison of ¹H and ¹³C NMR data with that reported in the literature. Response factors were calculated using the equation RF=DR/C, where DR was the detector response in peak area (PA) and C was the sorgoleone concentration. Confirmed integrated peaks were used to determine the percentage of sorgoleone in the extract or plant tissue. The RF of the target chemical constituent was used to determine the “percent” for each sample using the equation: PA/RF/C×100=% (peak area/response factor/concentration) in the plant tissue.

### Root Hair Isolation and RNA Extraction

Root hairs were isolated from dark-grown 7-day-old Sorghum seedling root systems as previously described (Baerson et al, 2008, supra), involving immersion in liquid nitrogen with gentle stirring, followed by filtration through a 90 μm mesh (Gilson Company, Inc. Lewis Center, OH, USA) to remove root system debris. Purity of the root hair preparations was assessed by bright field microscopy, and only highly enriched preparations were retained for subsequent RNA extraction. Total RNAs were isolated from root hairs using a Quick-RNA Microprep Kit (Zymo Research, Irvine, CA, USA) per the manufacturer's instructions. RNA purity was determined spectrophotometrically, and integrity was assessed using an Agilent 2100 Bioanalyzer (Agilent Technologies, Santa Clara, CA, USA).

### RNA-Seq and Data Analysis

Sequencing libraries were generated with 300 ng to 1 μg of RNA using the TruSeq Stranded mRNA Sample Preparation Kit (Illumina, San Diego, CA, USA). The libraries were assessed for insert size and purity using an Agilent 2100 Bioanalyzer, and quantitated by RT-qPCR using a KAPA Biosystems Library Quantitation kit (Roche, Pleasanton, CA, USA). Libraries were normalized, pooled, and diluted to a loading concentration of 1.8 pM, loaded on an Illumina High-Output Flow Cell, and sequenced using an Illumina NextSeq 500 instrument (Illumina, San Diego, CA, USA). The sequencing run generated ˜20 million 150 bp paired-end reads per sample, and all run metrics, including Q-Score distributions, cluster densities, and total sequence yields, were within the recommended parameters.

Data analysis was performed using the Qiagen CLC Genomics Workbench (CLCGxWB) version 21 software. Sequence reads were mapped to the S. bicolor reference genome (Sbicolor_454_v3.1.1. genome; phytozome-next.jgi.doe.gov/info/Sbicolor_v3_1_1), using default parameters with a strand-specific alignment protocol. The mapping report indicated that all metrics fell within the recommended parameters, with >90% of the reads per sample mapping to the reference genome. The mapping results generated values for Total Counts, Transcripts per Million reads, as well as Fragments Per Kilobase of exon per Million reads mapped (FPKM) for each gene. The “Differential Expression for RNA-Seq” tool in CLCGxWB software was used with default parameters to identify differentially expressed genes across samples. Genes that had a fold-change of ≥2, an FDR-corrected p-value of <0.05, and a Maximum Group Mean FPKM of >2 (i.e., the maximum of the average FPKM across the two groups in each statistical comparison) were considered to be significantly differentially expressed. Hierarchical cluster analysis was performed in CLCGxWB software as well, using the Euclidean distance and complete linkage clustering method. Gene ontology (GO) enrichment analysis was conducted using GO-based molecular function categories available at the agriGO v2.0 website (systemsbiology.cau.edu.cn/agriGOv2/).

### Phylogenetic Tree and Ortholog Detection

Phylogenetic tree construction and ortholog predictions were performed using the SHOOT phylogenetic gene search and ortholog inference tool (www.shoot.bio/). The resulting tree was visualized with phylotree.js. Ortholog analyses were performed using the “plants database” option in SHOOT.

### Results and Discussion

### Utilization of Genotype Tx430 as a Model for Sorgoleone Transport Studies

The allelochemical sorgoleone is synthesized predominantly or exclusively in root hair cells (Weston et al., 2013, supra). It is therefore reasonable to speculate that transporters and other transport components required for sorgoleone rhizosecretion could primarily be localized to root hair cells, while noting the possibility that specific subunits within heteromeric complexes may exhibit non-root hair specific expression. We further speculated that the sorgoleone biosynthetic pathway could be subject to feedback regulation as is the case for many plant specialized metabolic pathways (Li et al., (2023), supra), thus the identification of candidate transporter sequences responsive to changes in sorgoleone pathway flux could also be of particular interest. A transcriptomics and reverse genetics-based approach was therefore pursued, taking advantage of the availability of sorgoleone-deficient RNAi transgenic S. bicolor (genotype Tx430) lines (Cook et al, (2010), supra).

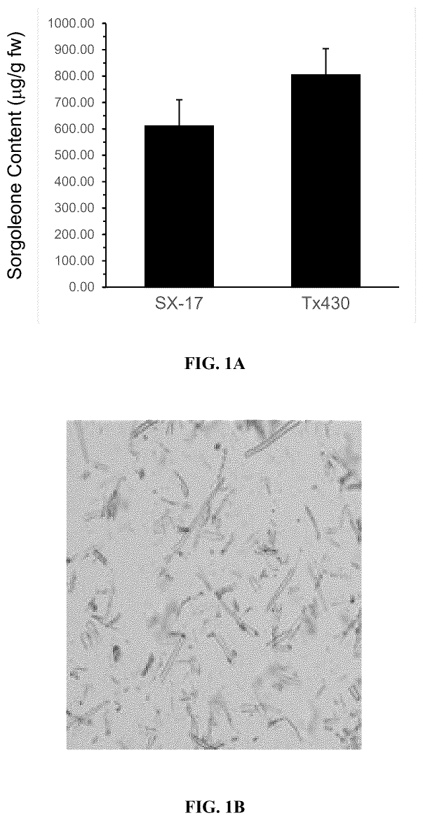

To assess the suitability of genotype Tx430 for use in these studies, sorgoleone content was first determined by HPLC analysis of root exudates collected from 7-day-old, etiolated seedlings. For comparison, sorgoleone content was also determined for SX-17 seedlings, a S. bicolor×S. sudanense hybrid frequently used for sorgoleone studies (e.g., Czarnota et al, (2003), supra). The results of this comparison are shown in FIG. 1A. The total sorgoleone content of approximately 600 μg/g fw measured in SX-17 seedlings is in agreement with previously reported levels for this genotype and was approximately 25% lower than that observed for Tx430 seedlings (FIG. 1A). Significant variation in sorgoleone content among different accessions is known to occur (Nimbal et al., (1996), supra; Czarnota et al., (2003), supra), however the HPLC analyses results revealed that the sorgoleone content of genotype Tx430 is comparable to levels observed in other accessions.

To further examine the utility of genotype Tx430, approximately 120 seedlings were grown for 1 week in aeroponic containers, and roots systems were then harvested, and root hairs isolated using a small-scale isolation procedure adapted from previously published methods (Baerson et al., (2005), supra). Highly enriched root hair preparations were readily obtained from the Tx430 seedlings grown under these conditions (FIG. 1B), and in sufficient quantities for the generation of sequencing libraries for RNA-seq studies (data not shown). Taken together, these results indicate the suitability of the genotype Tx430 as a model system for identifying genes associated with sorgoleone extracellular transport.

Identification of Transporter-like Sequences Preferentially Expressed in Root Hairs

As mentioned, sorgoleone biosynthesis occurs primarily or exclusively in root hairs, therefore RNA-seq studies were employed to identify putative transporter sequences expressed predominantly within this cell type. Total RNAs were prepared from S. bicolor isolated root hairs, root systems, developing panicles, stems, immature and fully expanded leaves and shoot apices, and used for the construction of Illumina TruSeq sequencing libraries. Approximately 20×10⁶ quality reads were generated from each library, with each tissue type analyzed in triplicate. From these analyses, 1,126 sequences with expression ≥2-fold higher in root hairs than all other tissues analyzed were identified, and 224 sequences with expression levels 2-fold lower in root hairs than all other tissues were also found (data not shown). Significantly, 139 sequences potentially encoding diverse cellular transporters and putative lipid transfer proteins were identified among the 1,126 sequences exhibiting root hair-preferential expression. As expected, the gene ontologies for this subset were highly enriched for transporter-related functions. In addition, the GO category ‘ATPase activity’ was highly significant within this group, which would be predicted given the prominent role ATP hydrolysis plays in numerous transport-related processes (Do et al., (2021), supra).

### Transcriptional Feedback Control of Sorgoleone Biosynthesis

Specialized metabolic pathways frequently employ feedback regulatory mechanisms as a means for modulating pathway flux in response to changing environmental and cellular conditions (Li et al., (2023), supra). If sorgoleone biosynthesis and transport were regulated in this manner at the transcriptional level, then monitoring changes in transcript abundance in response to changes in pathway flux could potentially provide an additional criterion for the selection of candidate transporter sequences. To explore this possibility, RNA-seq experiments were conducted with root hairs isolated from S. bicolor genotype Tx430 seedlings transformed with a binary vector designed for RNAi-mediated down-regulation of SbARS1 and SbARS2, which exhibit dramatically reduced sorgoleone contents relative to non-transformed Tx430 seedlings (Cook et al., (2010), supra). Root hairs isolated from wild-type Tx430 seedlings and null (non-transformed) segregants from the same transgenic event grown in parallel under identical conditions were also used for RNA-seq analyses, and sequences differentially expressed between wild-type vs. sorgoleone-deficient seedlings, and null segregant vs. sorgoleone-deficient seedlings were identified. The RNA-seq datasets were further mined for additional putative transporter sequences using gene ontologies, and conserved motifs derived from the SMART, Panther, Prosite and PFAM databases using the BIOMART tool (phytozome-next.jgi.doe.gov/biomart).

As shown in Table 1, the RNA-seq analyses revealed that transcripts representing all of the biosynthetic enzymes known to be involved in sorgoleone biosynthesis were significantly down-regulated in the sorgoleone-deficient transformed seedlings. Transcript levels for SbDES2 and SbDES3, the fatty acid desaturases which sequentially convert C16:0 palmitoyl-CoA to the C16:3 hexadecatrienoyl-CoA (Pan et al., (2007), supra) were observed to be approximately 4.5 to 5.4-lower in sorgoleone-deficient seedlings, relative to wild-type and null segregant controls (Table 1). Transcript levels for SbOMT3, which catalyzes the O-methylation of the 5-pentadecatrienyl resorcinol intermediate (Baerson et al., (2008), supra), were decreased by more than 4-fold relative to control seedlings. Transcripts for SbCYP71AM1, the P450 enzyme catalyzing the dihydroxylation of the 5-pentadecatrienyl resorcinol-3-methyl ether (Pan et al., (2018), supra), were decreased by approximately 2.5-fold in the sorgoleone-deficient seedlings. The alkylresorcinol synthases participating in the formation of the pentadecatrienyl resorcinol intermediate (Cook et al., (2010), supra), were specifically targeted for RNAi-mediated inhibition, thus potential effects of sorgoleone-deficiency on SbARS1, SbARS2 and SbARS3 expression are not directly discernible.

TABLE 1
Transcriptional Feedback Response
of Sorgoleone Biosynthetic Genes

| | Expression (fold change) | | | |
|---|---|---|---|---|
| | 274+ vs 274 | | 278+ vs 278 | |
| Gene | null | 274+ vs WT | null | 278+ vs WT |
| DES2 | −3 | −6.3 | −5.4 | −4.7 |
| DES3 | −2.5 | −5.4 | −4.9 | −4.5 |
| ARS1 | −10.2 | −24 | −12.2 | −19.9 |
| ARS2 | −28.2 | −59.4 | −39.6 | −35.1 |
| ARS3 | −21.8 | −42.3 | −70.1 | −58.2 |
| OMT3 | −2.5 | −5.2 | −4.3 | −4.4 |
| CYP71AM1 | | | −2.5 | −2.7 |

Taken together, these results would be consistent with transcriptional feedback repression of the sorgoleone pathway occurring under conditions of reduced pathway flux. The possibility cannot be discounted therefore that cellular transporters involved in sorgoleone rhizosecretion could be similarly affected under conditions of reduced sorgoleone biosynthesis. This form of feedback control could potentially involve sorgoleone pathway intermediates or other small molecules used as ‘signals’ to detect perturbations in sorgoleone pathway flux, or in pathways interconnected with sorgoleone biosynthesis, leading to the activation of an inhibitory signaling cascade. An analogous situation may occur in Arabidopsis thaliana, where phenylpropanoid pathway intermediates are thought to act as signals playing a role in the maintenance of phenylpropanoid homeostasis, and perturbations in phenylpropanoid metabolism lead to a repression of lignin biosynthesis. Genetic analyses revealed the involvement of the MED5a and MED5b Mediator complex subunits in this inhibitory signaling cascade (Bonawitz et al., (2014), supra).

Identification of a Full-Length ABC Subfamily G Protein Member Required for Sorgoleone Rhizosecretion

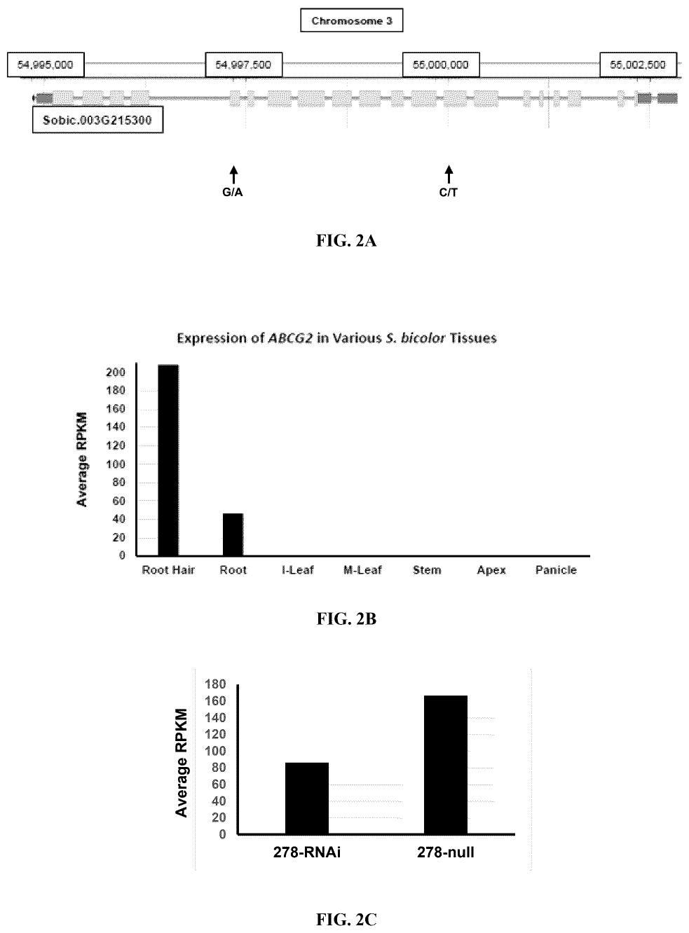

Candidate transporter-like sequences exhibiting root hair preferential expression patterns were prioritized for follow-up analyses, and expression levels observed in sorgoleone-deficient transgenic vs. wild-type or null segregant control seedlings were also taken into account. Priority was also placed on ABC transporter-like sequences, particularly ABCG subfamily members, given their prominent role in the transport of plant lipidic compounds (Do et al., (2021), supra). One candidate sequence, Sobic.003G215300 located on chromosome 3 (S. bicolor v3.1.1 genome; phytozome-next.jgi.doe.gov), corresponding to a putative full-length ABCG subfamily transporter (FIG. 2A), exhibited highly root hair-preferential expression (FIG. 2B). Average RPKM values for the corresponding transcript were approximately 8-fold higher in isolated root hairs than total seedling root systems and were extremely low (mean RPKM <5) in panicles, shoot apices, stems, and mature and immature leaves (FIG. 2B). It should also be considered that the total root tissues used for RNA-seq were harvested from seedlings grown under conditions identical to those used for root hair preparations and thus contained extensive amounts of root hairs, therefore root hairs likely contributed a significant percentage of the transcripts detected in the total root samples. Of further significance, the RNA-seq analyses also revealed that Sobic.003G215300 expression levels decreased approximately two-fold in sorgoleone-deficient seedlings (FIG. 2C), thus this sequence encodes an ABCG subfamily transporter potentially co-regulated with SbDES2, SbDES3, SbOMT3, SbCYP71AM1 and possibly SbARS1-3 (Table 1).

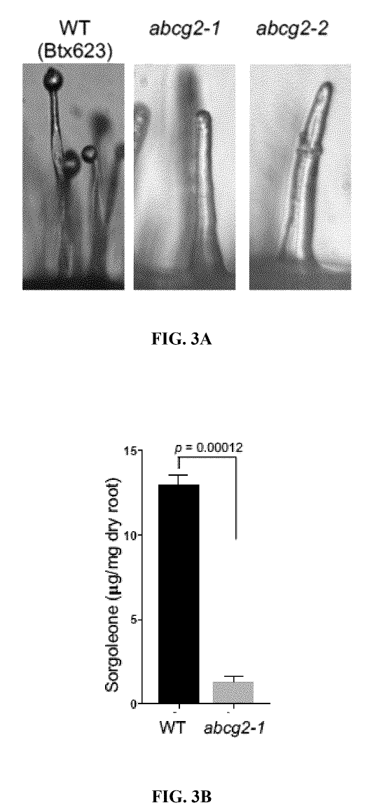

To further examine the potential involvement of Sobic.003G215300 in sorgoleone rhizosecretion, publicly available S. bicolor mutant populations were searched, and two independent ems-generated nonsense mutant alleles were identified from genotype BTx623 (Jiao et al., (2016), supra). One of the identified nonsense mutants possesses a C→T nucleotide transition creating a stop codon at residue 338 (FIG. 2A; variant ID no. tmp_3_55000034_C_T; ensembl.gramene.org/Sorghum_bicolor) and the second possesses a G→A nucleotide transition creating a stop codon at residue 959 (FIG. 2A; tmp_3_54997363_G_A; ensembl.gramene.org/Sorghum_bicolor). As shown in FIG. 3A and FIG. 3B, both mutants exhibited greatly reduced secretory activity, visible as a substantial reduction of exudate droplet formation from root hairs (FIG. 3A). Importantly, HPLC analyses further revealed a dramatic loss of sorgoleone exudation from mutant seedlings, decreasing by more than 90% relative to wild-type genotype BTx623 controls (FIG. 3B). Additionally, co-segregation analyses revealed that the mutations co-segregated with the reduced secretion phenotype (data not shown). Taken together, these results demonstrate an in vivo role for the ABCG subtype transporter encoded by Sobic.003G215300 in the rhizosecretion of the allelochemical sorgoleone. This transporter encoded by this sequence is tentatively designated SbABCG2, and the mutant alleles used for these analyses as abcg2-1 and abcg-2 (FIG. 3A, FIG. 3B).

In principle, SbABCG2 is likely to play a dual role in S. bicolor root hairs: to facilitate the transport of sorgoleone to the rhizosphere where it can play a role defensive role, and as a primary component of the autotoxicity mechanism preventing Sorghum spp. members from succumbing to the inhibitory effects of the compound. Given that sorgoleone biosynthesis is restricted to Sorghum spp. members, SbABCG2 or other ABCG proteins with similar activity will likely be required for the deployment of sorgoleone as a plant-incorporated-protectant in other crop species that possess no natural tolerance to the allelochemical.

### Example 2

### Construction of a Transgenic Plant Expressing SbABCG2

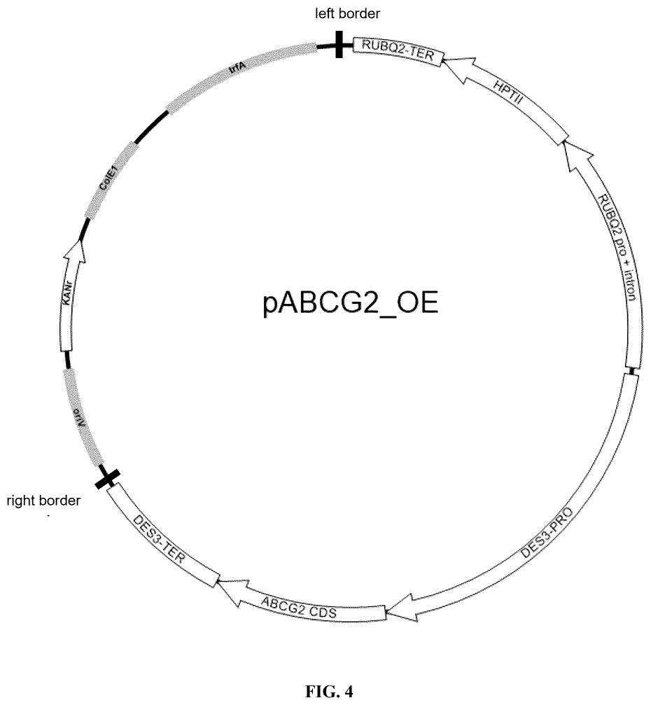

To produce transgenic plants overexpressing the S. bicolor ABC subtype G transporter ABCG2 (e.g., SEQ ID NO:1, as encoded by SEQ ID NO:2) in root hair cells, a binary vector is developed for the high-level expression of the ABCG2 sequence in planta (FIG. 4). For this approach, the complete open reading frame of ABCG2 is positioned downstream of the S. bicolor root hair-specific DES3 gene promoter (5′ flanking region) and its cognate 5′ UTR, and immediately upstream of the S. bicolor DES3 gene 3′ flanking region (see, e.g., U.S. Pat. No. 10,000,762). The ABCG2 overexpression transgene cassette is cloned within the T-DNA borders of a binary vector containing a hygromycin phosphatase II (HPTII) plant-selectable marker driven by the rice ubiquitin 2 gene promoter (5′ flanking region) containing a cognate intron located within the 5′ UTR region (Wang J, Oard J H. Rice ubiquitin promoters: deletion analysis and potential usefulness in plant transformation systems. Plant Cell Rep. 2003 September; 22(2):129-34.). The resulting construct, exemplified in FIG. 4, contains the ABCG2 expression cassette arranged in a head-to-head orientation with respect to the HPTII selectable-marker cassette, and is designated pABCG2_OE. All DNA manipulations involved in the construction of pABCG2_OE involve standard cloning procedures (Sambrook, et al, supra). Recombinant A. tumefaciens strains harboring pABCG2_OE are used to transform immature embryos of Sorghum bicolor (genotype Tx430) as previously described (Howe et al, Plant Cell Rep., (2006), 8:784-91), or Arabadopsis thalania, in which case an HPTII selectable marker cassette containing an enhanced CaMV 35S promoter (Kay et al, Science, (1987), 236:1299-1302.), and the CaMV 35S transcript polyadenylation region would be substituted. For the generation of Arabidopsis thaliana transformants, recombinant A. tumefaciens strains harboring the vector are used to transform Arabidopsis thaliana (cv. Col-0) using the ‘floral-dip’ procedure (Clough & Bent, Plant J., (1998), 16:735-43).

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. An expression vector comprising a promoter and a heterologous polynucleotide, wherein the heterologous polynucleotide encodes a sorgoleone transporter having an amino acid sequence at least 75% identical to SEQ ID NO: 1, and wherein the promoter is operatively linked to the heterologous polynucleotide.

2. The expression vector of claim 1, wherein the promoter is selected from the group consisting of a constitutive promoter, a tissue-specific promoter, and an inducible promoter.

3. The expression vector of claim 1, wherein the promoter is a root-hair-specific promoter.

4. The expression vector of claim 1, wherein the heterologous polynucleotide comprises a sequence at least 75% identical to SEQ ID NO: 2.

5. A transformed cell, comprising the expression vector of claim 1, wherein said transformed cell is selected from the group consisting of a plant cell, a fungal cell, a blue-green alga, and a bacterial cell.

6. The transformed cell of claim 5, wherein the plant cell is not a Sorghum bicolor cell.

7. A transgenic organism comprising the transformed cell of claim 5; wherein said transgenic organism produces a sorgoleone transporter with at least 75% identity to SEQ ID NO: 1.

8. The transgenic organism of claim 7, wherein the organism is not Sorghum bicolor.

9. The transgenic organism of claim 7, wherein the organism is a plant.

10. A transgenic plant comprising a heterologous nucleic acid encoding a protein having at least 75% identity to SEQ ID NO: 1.

11. The transgenic plant of claim 10, wherein the plant is not Sorghum bicolor.

12. A method of expressing a sorgoleone transporter in a plant or plant cell, comprising the steps of introducing a heterologous nucleic acid into a genome of the plant or plant cell, wherein the heterologous nucleic acid is operably linked to a promoter functional in the plant or plant cell, wherein the promoter is a constitutive promoter, a tissue-specific promoter, or an inducible promoter, wherein the heterologous nucleic acid encodes a protein at least 75% identical to SEQ ID NO:1 and the protein has sorgoleone transport activity, and allowing the production of the sorgoleone transporter in the plant or plant cell.

13. The method of claim 12, wherein the promoter is a tissue-specific promoter.

14. The method of claim 13, wherein the tissue-specific promoter is a root-hair specific promoter.

15. The method of claim 11, wherein the plant or plant cell is not Sorghum bicolor.

16. A method of generating a sorgoleone-resistant plant, comprising the steps of introducing a heterologous nucleic acid into a genome of the plant, wherein the heterologous nucleic acid is operably linked to a promoter functional in the plant wherein the promoter is a constitutive promoter, a tissue-specific promoter, or an inducible promoter, wherein the heterologous nucleic acid encodes a protein at least 75% identical to SEQ ID NO:1 and the protein has sorgoleone transport activity, and allowing the production of the sorgoleone transporter in the plant, thereby generating a sorgoleone-resistant plant.

17. The method of claim 16, wherein the promoter is a tissue-specific promoter.

18. The method of claim 17, wherein the tissue-specific promoter is a root-hair specific promoter.

19. A method of controlling weeds in a field, comprising the steps of planting the transgenic plant of claim 10 in the field, and applying sorgoleone to the field such that it contacts the weeds, thereby controlling sorgoleone-susceptible weeds.


SEQUENCE LISTING (2 sequences)

SEQ ID NO: 1 - protein, 1281 aa, Sorghum bicolor
MPILHDVSGIVKPCRMTLLLGPPGSGKTTLLLALAGRLHNNLKVSGKVTYNGHEMDEFVPERTAAYISQHDLHIGEMTVRETLEFSARCQGVGTRFDLLAELSRREKAGNIKPDTDIDAFMKACSMRGQEANVICDYILKILGLEICADTMVGDEMLRGISGGQRKRVTTGEMLVGPANALFMDEISTGLDTSTTFQIIKSIRQTIHILGGTALISLLQPAPETYDLFDDIILLSDGQIVYQGPRESVLEFFLSLGFKCPQRKGVADFLQEVTSRKDQKQYWVWHDKPYRYVSVKEFASAFQSFHVGRAVAHELAIPFDKSKNHPGALTTSRYGVSAWELFKANVDRELLLMKRNSFVYIFRTLQLMITTIIVMTLFFRTNMHRDSVTDGGIYMGALFFSVLLIMLNGFSELALTIMKIPVFFKQRDLLFFPAWAYTIPTWILKIPISFIEVGGFVFMAYYVIGFDPNVVRFFKQYLLFLAVNQMAAALFRFIGGAARDMTVANVFGSFVLLIFMVLCGFILDREKVKKWWIWGYWISPMMYAQNALSVNEMLGHSWDKILNSSMSNETLGVQSLKSRGIFPEAKWYWIGLAALIGFVMLFNCLFTLALAYLKPYGKSHPSISEEELKAKYANINGNVVAEDSLPVGSSHLETVGITRSSSATVENHSGTMQRGMILPFAPLSLTFSNIKYFVDMPQEMKTHGVVGDRLELLKGISGSFRPGVLTALMGVSGAGKTTLMDVLAGRKTSGYIEGNISISGYPKKQETFARVSGYCEQNDIHSPHVTVYESLVFSAWLRLPTDVDSNTRKMFIEEVMELVELKPLRNALVGLPGVNGLSTEQRKRLTIAVELVANPSIIFMDEPTSGLDARAAAIVMRTVRNIVDTGRTIVCTIHQPSIDIFEAFDELFLMKRGGEEIYVGPLGHHSSELIKYFEGIEGVKKIEDGYNPATWMLEVTAVSQEQILGVDFSDLYKKSELYQRNRALIQELSEPPAGSSDLHFHSQYAQSFFMQCLACLWKQNLSYWRNPAYNAVRLFFTTVIALMFGTIFWDLGGKMGQPQDLFNAMGSMYAAVMFIGVLNSTSVQPVVSVERTVFYRERAAGMYSALPYAFGQVSIELPYILVQAIVYGIIVYSMIGFEWTVAKLFWYLFFMYFTFLYFTFYGMMAVGLTPSYHVAAIVSTLFYGIWNLFSGFLIPLPKVPIWWKWYCWACPVAWSLYGLVVSQFGDIRTPMDDGVPVNVFVENYFDFKHSWLGVVAIVVVAFVVLFAFLFGFAIMKLNFQRR

SEQ ID NO: 2 - DNA, 3846 bp, Sorghum bicolor
  source 1..3846: mol_type = other DNA
atgccgatcctgcacgacgtcagcggcatcgtcaagccctgcagaatgactctgctgttaggtccgcccgggtcagggaaaaccaccttgctgctcgccttggccgggaggctccataacaacctcaaggtttcaggcaaagtgacctacaatgggcatgaaatggatgagtttgtgcctgagaggacagccgcgtatatcagccagcatgaccttcatattggagagatgactgtcagagagacgcttgagttctctgcacgctgtcaaggtgttggcactcgtttcgatttgttggctgagctttcaaggcgggagaaggccggaaatataaaacctgataccgatatcgatgcattcatgaaggcttgttcaatgcgaggacaggaagctaatgtgatctgtgactatatattgaagatattaggattagaaatatgtgctgacacgatggtgggagatgagatgttgaggggcatctctgggggacaacggaagcgtgtcacaacaggcgagatgcttgttggtccggccaatgcactgttcatggatgaaatttcaactgggcttgacacctccactacgttccagatcattaagtcgattagacagacaatccacatcctcggtggcacagctctcatctccttgttacagcctgcacctgagacatatgatctttttgatgacatcatactcctctcagacgggcagattgtgtatcaggggccccgagaaagtgtgcttgaattcttcttgtctctagggttcaagtgtcctcagagaaagggtgtggctgactttttgcaagaagtgacttccaggaaagatcagaagcaatactgggtgtggcacgacaagccgtaccgatatgtgtcagttaaggaatttgcatccgcattccagtctttccatgttgggagagctgtagcacatgagctagccattccgttcgacaagagcaaaaaccatcccggtgctctgaccacctcaaggtatggtgtcagtgcctgggagctattcaaagcaaacgtcgatcgagaactcttgcttatgaagaggaattccttcgtctacatctttagaactttgcagctgatgataacgaccataattgtaatgactctcttctttcgtaccaatatgcaccgtgactcagtgactgatggtggtatctacatgggtgcgctcttcttttcggtgctcttgatcatgttgaatggattttctgagcttgcactcaccatcatgaagataccagtgttcttcaagcagagagatctcctcttctttccagcatgggcctacaccatacccacatggatcctcaagatcccaatctcttttattgaggttggtgggtttgttttcatggcatactatgtcatcgggtttgacccaaatgtagtcaggttcttcaagcaatatctcctatttttagcagtcaatcagatggcagcagcactcttccgatttattggtggggcagcaagggacatgactgttgcaaacgtctttggatcattcgtgctgctaatctttatggtgttgtgtggatttattctagatagagaaaaagtgaagaagtggtggatatggggatattggatctccccaatgatgtatgcccagaatgccctctcagtaaatgagatgctagggcatagttgggacaaaattttgaatagctctatgtccaatgaaaccctaggtgtgcaatccctgaagtcccgcggaattttcccagaagcaaagtggtattggatcggcttagctgcattgattggatttgtcatgcttttcaattgtctcttcacgcttgccctagcgtacctgaagccatatggcaagtcccatccgtctatatctgaagaggaactgaaggcgaagtatgcaaacatcaatggtaatgttgtggctgaagacagcttgccagtaggaagtagccacttagaaacagttggcatcactagatccagttcagcaactgttgaaaatcattctggcactatgcaaaggggtatgattcttccgtttgccccgctttcactgactttcagtaacatcaagtactttgttgacatgccccaggagatgaaaacacatggtgtagtcggggaccggttagagctcctcaaaggtatcagtgggtctttcagacctggagtgttgaccgcactgatgggtgttagtggtgctggcaagacaactctaatggatgtattggctgggagaaagaccagtggttacattgaaggaaacattagcatttctggatacccaaagaagcaagagacctttgcacgtgtatcaggatactgtgagcagaatgatatccactcaccgcacgtgacagtctatgagtcacttgttttctcagcatggctccgccttcctacagatgtagattcaaacacgagaaagatgttcattgaggaggtcatggaacttgtggagctcaagccattgagaaatgctttggttggacttcctggtgtaaatggtctgtcaactgaacagaggaaaagactgaccattgctgtggagctagttgctaatccctcaatcattttcatggatgagccaacctctggtcttgatgcccgagcagctgcaattgttatgaggacagtgaggaatattgttgatacaggtagaactatagtctgcacgattcatcagcctagcattgatatatttgaagcattcgatgagcttttccttatgaaacgaggaggagaggagatctatgttggtccactaggccatcattcgtctgagctgattaagtattttgagggaatcgagggagtcaagaaaattgaagatggctacaatccagcaacatggatgttagaagtaacagcagtttcgcaagaacaaatactaggtgttgattttagtgacttgtacaagaaatctgaattgtaccagaggaacagggccttgatacaagagttaagtgaaccaccagcaggctcaagtgaccttcatttccatagccagtacgcacagtctttcttcatgcaatgcctggcttgcttgtggaagcagaacctgtcatattggaggaaccctgcttacaatgccgttaggctatttttcacaacagtcatagcgcttatgtttggcaccatcttctgggaccttggtggtaaaatgggtcaaccacaagatttgttcaatgctatggggtccatgtatgctgcagtgatgttcatcggcgtcttgaattctacatctgtccagccagtagtctctgtggagcggaccgtgttttaccgcgaaagagctgccgggatgtactcggctttgccatatgcatttggacaggtttccattgaactcccatacattctggttcaggccatcgtatacgggatcatagtgtactccatgatcgggttcgagtggacagttgccaaattattttggtacctcttcttcatgtacttcacctttctctacttcacgttctatggaatgatggctgtcggcttgacgccgagctaccatgttgcggcgatcgtctccacactgttctatggtatctggaacctcttctctggattcttgatccctctccccaaagtcccgatctggtggaagtggtactgttgggcatgccctgtggcatggtcgctgtacgggctggttgtgtctcagtttggggacatcaggacgcccatggatgacggtgtccccgtgaatgtgttcgtggagaattacttcgacttcaagcacagctggttgggcgtggtggccatagtggtcgtggcgttcgtcgtcctctttgctttcctattcggctttgccatcatgaagctcaacttccaaaggagatag